(19) United States
(12) Patent Application Publication
(10) Pub. No.: US 2017/0135853 A1
(43) Pub. Date: May 18, 2017

(54) Far Infrared Hot Air Type Therapeutic Apparatus with Multiple Functions

(71) Applicant: Welter's Co., Ltd., Yinlin (TW)

(72) Inventor: Walter W. Hsu, Yinlin (TW)

(21) Appl. No.: 14/944,305

(22) Filed: Nov. 18, 2015

(51) Int. Cl. A61F 7/00; A61M 37/00

(52) U.S. Cl. CPC A61F 7/007; A61M 37/00; A61F 2007/0006; A61F 2007/0017; A61F 2007/0046; A61F 2007/006; A61F 2007/0086; A61F 2007/0088; A61M 2205/505; A61M 2205/75; A61M 2205/3368

(57) ABSTRACT

A therapeutic apparatus includes a housing (40), a circuit module (20) mounted in the housing, an electric heating element (10) mounted in the housing and electrically connected with the circuit module, an air delivery and regulating unit (30) mounted in the housing and electrically connected with the circuit module, and a control unit (50) mounted on the housing and electrically connected with the circuit module, the housing and the air delivery and regulating unit. Thus, the therapeutic apparatus has a sterilization effect and has the effect of blood circulation and cell activation.

### BACKGROUND OF THE INVENTION

1. Field of the Invention

The present invention relates to a therapeutic apparatus and, more particularly, to a far infrared hot air type therapeutic apparatus.

2. Description of the Related Art

A conventional therapeutic apparatus in accordance with the prior art shown in FIG. 1 comprises a housing 1 having a plurality of inlet holes 2, a filtering net 3 mounted in the housing 1 and aligning with the inlet holes 2, a flow channel 4 mounted in the housing 1 and having a first end aligning with the inlet holes 2 and a second end protruding outward from the housing 1, a connecting pipe 5 connected with the second end of the flow channel 4, an electric heater 8 mounted in the housing 1 and having an electric heating filament 7 extending into the flow channel 4, a fan 6 mounted in the flow channel 4 and disposed between the inlet holes 2 and the electric heating filament 7. In operation, ambient air in introduced through the inlet holes 2 into the flow channel 4 by operation of the fan 6. Then, the electric heating filament 7 heats the air to form a hot air flow which is blown by the fan 6 to pass through the connecting pipe 5 into an affected part of a user, such as the nose or mouth of the user so as to cure the affected part of the user. The heat or temperature from the electric heating filament 7 is controlled by a temperature regulator. However, when the hot air flow is blown outward from the connecting pipe 5, the ambient condition outside of the housing 1 will affect the hot air flow, so that the temperature of the hot air flow is not controlled easily. In addition, micro dust in the air will mix with the hot air flow to be inhaled into the affected part of the user, thereby decreasing the curing effect. Further, the fan 6 is far spaced from the electric heating filament 7 to prevent scalding and reduce the temperature of the hot air flow, and the connecting pipe 5 has a long distance to release the high temperature of the hot air flow so that the conventional therapeutic apparatus has a larger volume and is not carried and operated easily.

### BRIEF SUMMARY OF THE INVENTION

The primary objective of the present invention is to provide a far infrared hot air type therapeutic apparatus with multiple functions.

According to the primary advantage of the present invention, the therapeutic apparatus has a sterilization effect, has the effect of blood circulation and cell activation, and has the effect of hot cure and cell recovery.

According to another advantage of the present invention, the therapeutic apparatus is available for different affected parts of the human body, such as the nose, the mouth and the toes.

According to a further advantage of the present invention, the therapeutic apparatus has a straining and dustproof function to protect the affected part of the human body.

Further benefits and advantages of the present invention will become apparent after a careful reading of the detailed description with appropriate reference to the accompanying drawings.

### BRIEF DESCRIPTION OF THE SEVERAL VIEWS OF THE DRAWING(S)

FIG. 1 is a cross-sectional view of a conventional therapeutic apparatus in accordance with the prior art.

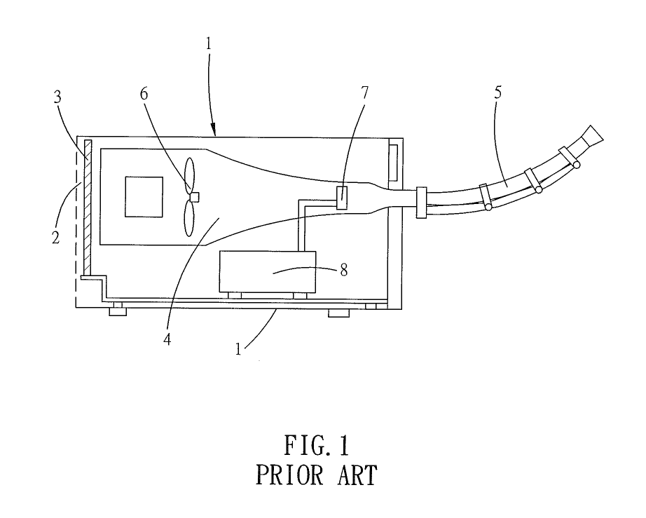

FIG. 2 is a perspective view of an electric heating element of a therapeutic apparatus in accordance with the preferred embodiment of the present invention.

FIG. 3 is a partially cross-sectional view of the therapeutic apparatus in accordance with the preferred embodiment of the present invention.

FIG. 4 is a circuit layout of the therapeutic apparatus in accordance with the preferred embodiment of the present invention.

FIG. 5 is a block diagram showing functions of the therapeutic apparatus in accordance with the preferred embodiment of the present invention.

FIG. 6 is a schematic operational view of the therapeutic apparatus as shown in FIG. 5 in use.

FIG. 7 is a partially cross-sectional view of the therapeutic apparatus in accordance with another preferred embodiment of the present invention.

FIG. 8 is a partially cross-sectional view of the therapeutic apparatus in accordance with another preferred embodiment of the present invention.

FIG. 9 is a partially cross-sectional view of the therapeutic apparatus in accordance with another preferred embodiment of the present invention.

FIG. 10 is a partially cross-sectional view of the therapeutic apparatus in accordance with another preferred embodiment of the present invention.

FIG. 11 is a partially cross-sectional view of the therapeutic apparatus in accordance with another preferred embodiment of the present invention.

FIG. 12 is a partially exploded perspective view of the therapeutic apparatus in accordance with another preferred embodiment of the present invention.

### DETAILED DESCRIPTION OF THE INVENTION

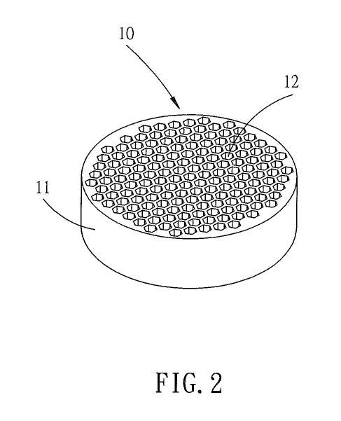

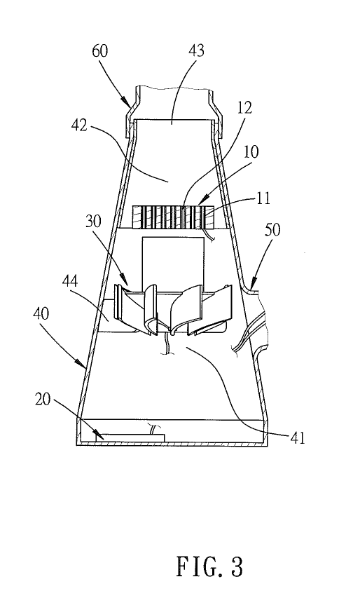

Referring to the drawings and initially to FIGS. 2-10, a therapeutic apparatus in accordance with the preferred embodiment of the present invention comprises a housing 40, a circuit module 20 mounted in the housing 40, an electric heating element 10 mounted in the housing 40 and electrically connected with the circuit module 20, an air delivery and regulating unit 30 mounted in the housing 40 and electrically connected with the circuit module 20, and a control unit 50 mounted on the housing 40 and electrically connected with the circuit module 20, the housing 40 and the air delivery and regulating unit 30. The therapeutic apparatus further comprises an atomized vapor humidification unit 70 electrically connected with the circuit module 20 and the control unit 50.

The housing 40 has a top provided with an air channel 42 which has an air outlet 43 and a bottom provided with an air chamber 41. The housing 40 is provided with a plurality of air inlets 44 each connected to the air chamber 41.

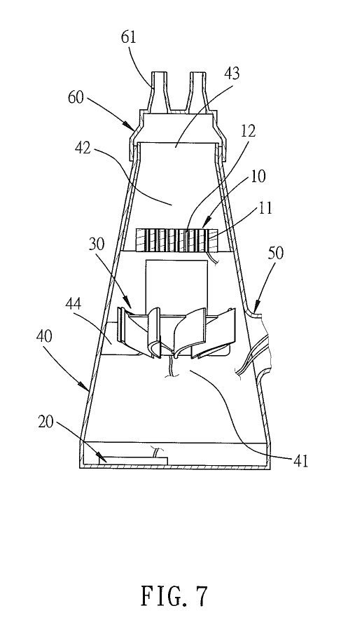

A fixture 60 is mounted on the air outlet 43 of the housing 40 and is provided with a contact head 61 (see FIG. 7). Preferably, the fixture 60 is made of soft heat resistant material, such as rubber.

The circuit module 20 includes a temperature detection and regulation circuit 22 and a rectifying circuit 24.

The electric heating element 10 is disposed between the air delivery and regulating unit 30 and the air outlet 43 of the housing 40 and includes an ceramic portion 11 with electrothermal and far infrared features. The ceramic portion 11 of the electric heating element 10 are made by ceramic nanometer powder and far infrared coating material which are combined together. The ceramic portion 11 of the electric heating element 10 has a porous structure and is provided with a plurality of through holes 12. The ceramic portion 11 of the electric heating element 10 has a multi-channel function by the through holes 12.

The air delivery and regulating unit 30 aligns with the air inlets 44 of the housing 40.

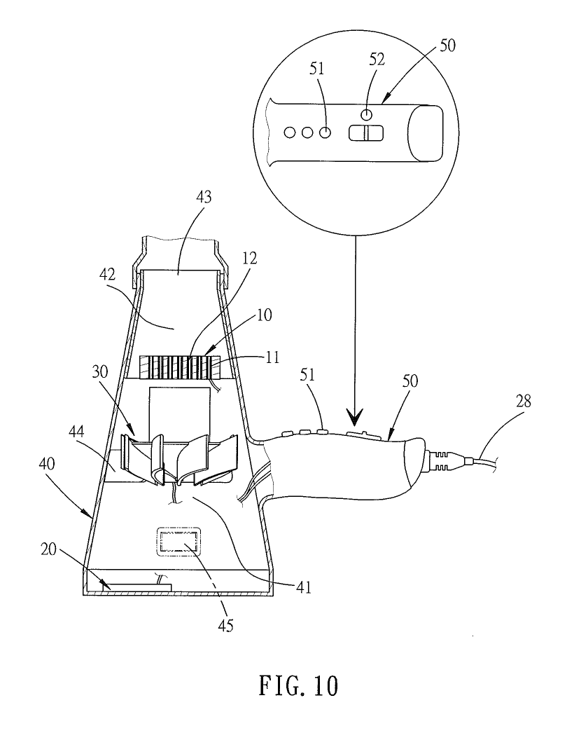

The control unit 50 extends outward from the housing 40 and is electrically connected with an external power supply 26 by an electric cord 28 (see FIG. 10). The control unit 50 includes a plurality of operation interfaces 51 (see FIG. 10), a power interruption switching button 52 (see FIG. 10) and a logic control and human machine interface 53. The control unit 50 controls and regulates the temperature of the electric heating element 10 by the temperature detection and regulation circuit 22 of the circuit module 20.

In use of the control unit 50, the operation interfaces 51 of the control unit 50 have a multi-channel temperature monitoring function by the through holes 12 of the electric heating element 10 and the temperature detection and regulation circuit 22 of the circuit module 20, a far infrared irradiating function by the ceramic portion 11 of the electric heating element 10, an atomized vapor humidifying function by the atomized vapor humidification unit 70, a touch control function by the logic control and human machine interface 53 and an emergency power interrupting function by the power interruption switching button 52. Thus, the operation interfaces 51 of the control unit 50 are operated to provide a temperature regulating function, to provide a multi-point temperature monitoring function, to calculate the optimum electric energy output, to evenly distribute the output power, and to control the temperature stably.

In practice of the control unit 50, when the control unit 50 is started to control functions of the operation interfaces 51, the electric heating element 10 is operated to emit a hot air flow at a predetermined temperature by actions of the temperature detection and regulation circuit 22 and the rectifying circuit 24 of the circuit module 20, and the hot air flow is transmitted by operation of the air delivery and regulating unit 30 to pass through the air channel 42 and the air outlet 43 of the housing 40 into the fixture 60, so that the hot air flow enters an affected part of a human body. Thus, the through holes 12 of the electric heating element 10 have a multi-channel function to form a evenly distributed air field, and the temperature detection and regulation circuit 22 of the circuit module 20 regulates the power of the electric heating element 10 to keep a stabilized temperature, so that the hot air flow is delivered to the affected part of the human body to achieve a sterilization effect. In addition, the electric heating element 10 is heated to increase the far infrared effect of the ceramic portion 11 so as to enhance blood circulation and activate cells of the human body.

In the preferred embodiment of the present invention, the ceramic portion 11 of the electric heating element 10 is heated and rectified by the circuit module 20 to have a predetermined temperature ranged between 25 and 150° C. In addition, the air delivery and regulating unit 30 introduces ambient cold air through the air inlets 44 into the air chamber 41 of the housing 40, and regulates the ambient cold air at a predetermined flow rate ranged between 0.1 and 5 L/min. Further, the electric heating element 10 has a hot source which heats the ambient cold air to a predetermined temperature ranged between 40 and 80° C. to provide a comfortable sensation to the user.

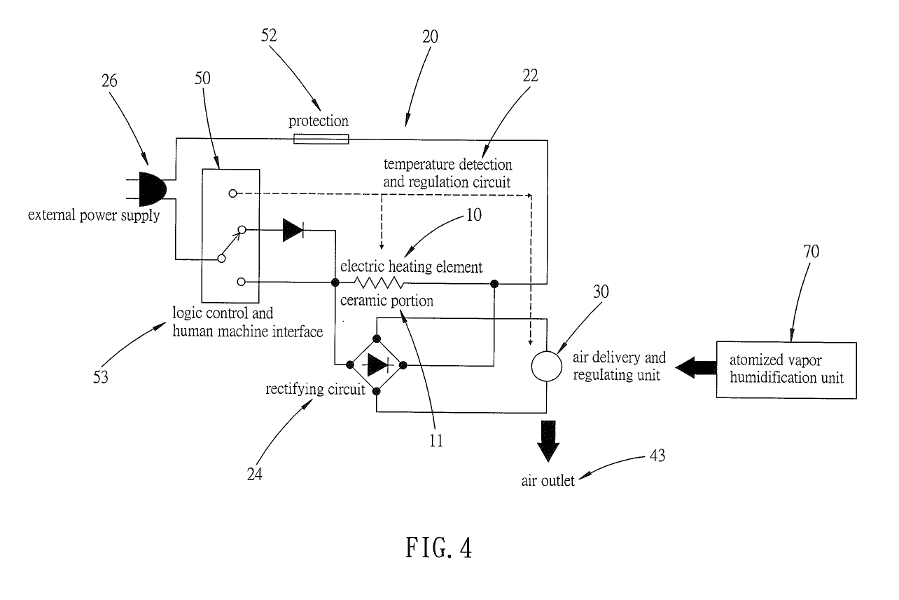

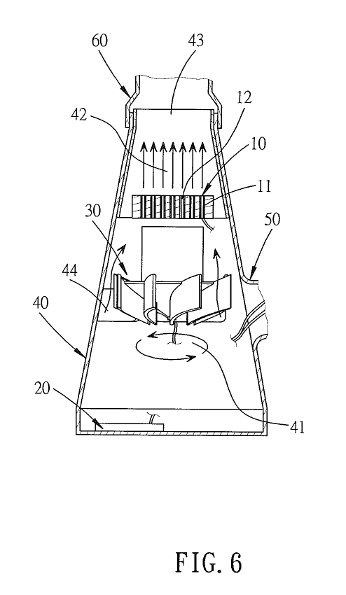

In operation of the therapeutic apparatus, referring to FIG. 6 with reference to FIGS. 2-4, the air delivery and regulating unit 30 introduces the ambient cold air through the air inlets 44 into the air chamber 41 of the housing 40, and regulates the ambient cold air at a steady flow rate ranged between 0.1 and 5 L/min. At this time, the air chamber 41 of the housing 40 under the air delivery and regulating unit 30 forms an air-blast effect to facilitate the air delivery and regulating unit 30 introducing the ambient cold air into the housing 40. Then, the electric heating element 10 heats the ambient cold air to form a hot air flow with a predetermined temperature ranged between 40 and 80° C. Then, the hot air flow is driven by operation of the air delivery and regulating unit 30 to flow through the air channel 42 and the air outlet 43 of the housing 40 into the fixture 60 as shown in FIG. 6, so that the hot air flow enters the affected part of the human body. At this time, the atomized vapor humidification unit 70 can co-operate with the air delivery and regulating unit 30 as shown in FIG. 4 so that the atomized vapor from the atomized vapor humidification unit 70 is mixed with the hot air flow from the electric heating element 10 to flow through the air channel 42 and the air outlet 43 of the housing 40 into the fixture 60.

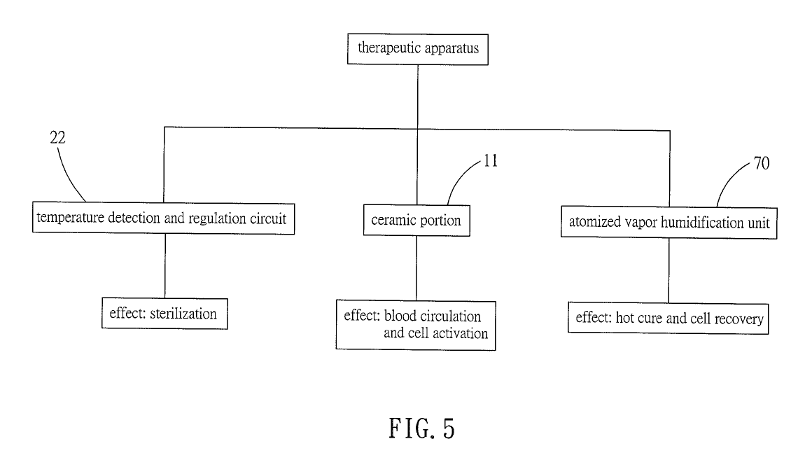

As shown in FIG. 5, the temperature detection and regulation circuit 22 of the circuit module 20 regulates the power of the electric heating element 10 to keep a stabilized temperature, so that the hot air flow from the air outlet 43 of the housing 40 is kept at a constant temperature to achieve a sterilization effect (the optimum sterilizing temperature is ranged between 40 and 45° C.). In addition, the electric heating element 10 is heated to increase the far infrared effect of the ceramic portion 11 so as to achieve the effect of blood circulation and cell activation. Further, the atomized vapor humidification unit 70 co-operates with the air delivery and regulating unit 30 so that the atomized vapor from the atomized vapor humidification unit 70 is mixed with the hot air flow from the electric heating element 10 so as to achieve the effect of hot cure and cell recovery.

As shown in FIG. 7, the contact head 61 of the fixture 60 is available for the nose of the human body.

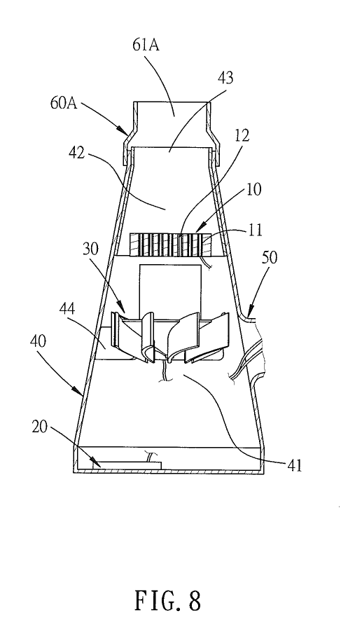

As shown in FIG. 8, the contact head 61A of the fixture 60A is available for the mouth of the human body.

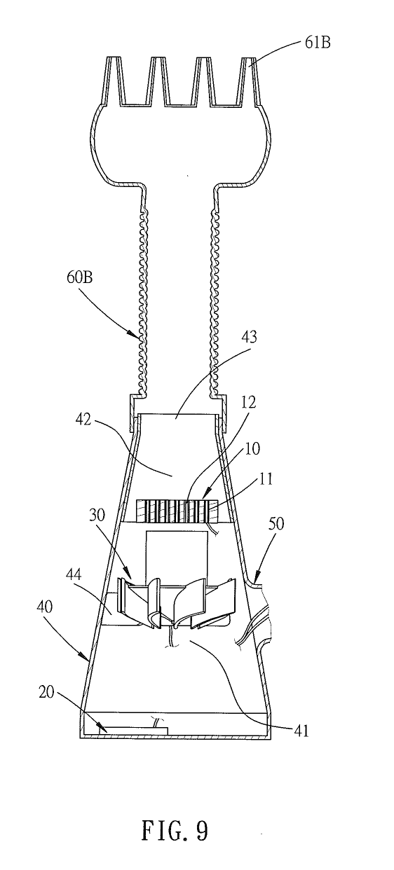

As shown in FIG. 9, the contact head 61B of the fixture 60B is available for the toes of the human body.

As shown in FIG. 10, the therapeutic apparatus further comprises a liquid crystal display panel 45 mounted on a peripheral wall of the housing 40 and electrically connected with the circuit module 20 and the control unit 50. The liquid crystal display panel 45 can indicate a safety temperature limit, and includes a touch control operation illustration interface, an audio and video convergence, a multi-language mode and an intellectual interaction introduction module.

When in use, the operation interfaces 51 of the control unit 50 are operated to set a predetermined temperature so that the ceramic portion 11 of the electric heating element 10 is driven by the circuit module 20 to produce a hot air flow which is kept at the predetermined temperature. In addition, the power interruption switching button 52 of the control unit 50 is operated to interrupt the electric power when the hot air flow exceeds the predetermined temperature, thereby preventing the user from being scalded. Further, the operation interfaces 51 of the control unit 50 are operated to function as a temperature control switch, to regulate the flow rate of the hot air flow and to control the action time. Further, the operation interfaces 51 of the control unit 50 are operated to select the affected part of the human body, such as the contact head 61 for the nose of the human body, the contact head 61A for the mouth of the human body and the contact head 61B for the toes of the human body. Further, the control unit 50 include a power switch to turn on/off a connection between the external power supply 26 and the therapeutic apparatus. Further, the logic control and human machine interface 53 of the control unit 50 facilitates the user operating the therapeutic apparatus. At the same time, the liquid crystal display panel 45 has operational selection items and has a sound interface that co-operates with the operation interfaces 51 of the control unit 50 to simultaneously sound the selected operational selection items and the operational function.

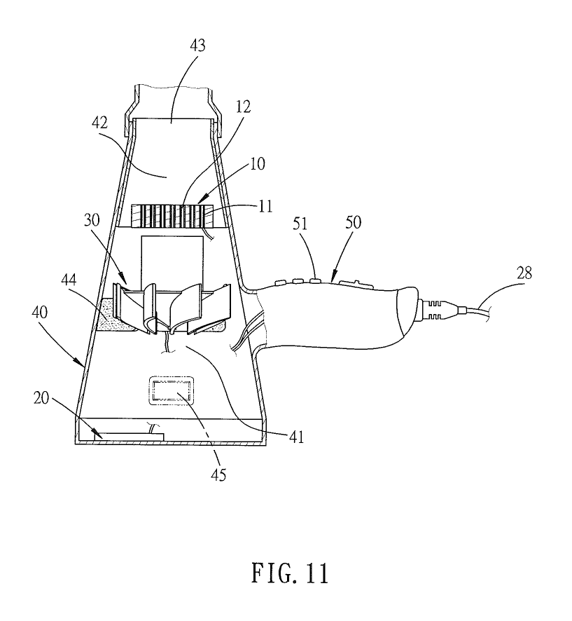

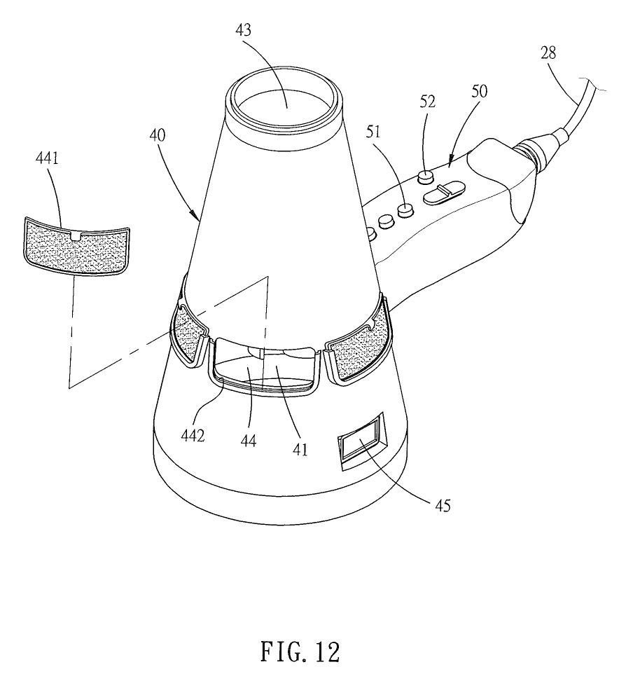

Referring to FIGS. 11 and 12, the therapeutic apparatus further comprises a plurality of filtering layers 441 mounted in the air inlets 44 of the housing 40 to provide a straining and dustproof function. Preferably, the filtering layers 441 are made of active carbon and are removably mounted in the air inlets 44 of the housing 40 to facilitate the user assembling and disassembling the filtering layers 441. Each of the air inlets 44 has a periphery provided with a mounting slot 442 for mounting the filtering layers 441.

Accordingly, the therapeutic apparatus has a sterilization effect, has the effect of blood circulation and cell activation, and has the effect of hot cure and cell recovery. In addition, the therapeutic apparatus is available for different affected parts of the human body, such as the nose, the mouth and the toes. Further, the therapeutic apparatus has a straining and dustproof function to protect the affected part of the human body.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

1. A therapeutic apparatus comprising:
a housing;
a circuit module mounted in the housing;
an electric heating element mounted in the housing and electrically connected with the circuit module;
an air delivery and regulating unit mounted in the housing and electrically connected with the circuit module; and
a control unit mounted on the housing and electrically connected with the circuit module, the housing and the air delivery and regulating unit;
wherein:
the housing has a top provided with an air channel which has an air outlet and a bottom provided with an air chamber;
the housing is provided with a plurality of air inlets each connected to the air chamber;
the circuit module includes a temperature detection and regulation circuit and a rectifying circuit;
the electric heating element is disposed between the air delivery and regulating unit and the air outlet of the housing and includes an ceramic portion with electrothermal and far infrared features;
the ceramic portion of the electric heating element is provided with a plurality of through holes;
the air delivery and regulating unit aligns with the air inlets of the housing;
the control unit extends outward from the housing and is electrically connected with an external power supply;
the control unit includes a plurality of operation interfaces;
when the control unit s started to control functions of the operation interfaces, the electric heating element is operated to emit a hot air flow at a predetermined temperature by actions of the temperature detection and regulation circuit and the rectifying circuit of the circuit module, and the hot air flow is transmitted by operation of the air delivery and regulating unit to pass through the air channel and the air outlet of the housing into a fixture, so that the hot air flow enters an affected part of a human body;
the through holes of the electric heating element have a multi-channel function to form a evenly distributed air field, and the temperature detection and regulation circuit of the circuit module regulates a power of the electric heating element to keep a stabilized temperature, so that the hot air flow is delivered to the affected part of the human body to achieve a sterilization effect;
the electric heating element is heated to increase the far infrared effect of the ceramic portion so as to enhance blood circulation and activate cells of the human body.

2. The therapeutic apparatus of claim 1, wherein:
the control unit further includes a power interruption switching button and a logic control and human machine interface;
the operation interfaces of the control unit have a multi-channel temperature monitoring function by the through holes of the electric heating element and the temperature detection and regulation circuit of the circuit module, a far infrared irradiating function by the ceramic portion of the electric heating element, an atomized vapor humidifying function by an atomized vapor humidification unit, a touch control function by the logic control and human machine interface, and an emergency power interrupting function by the power interruption switching button;
the control unit include a power switch to turn on/off a connection between the external power supply and the therapeutic apparatus;
the therapeutic apparatus further comprises a liquid crystal display panel mounted on a peripheral wall of the housing and electrically connected with the circuit module and the control unit;
the liquid crystal display panel can indicate a safety temperature limit, and includes a touch control operation illustration interface, an audio and video convergence, a multi-language mode and an intellectual interaction introduction module;
the liquid crystal display panel has operational selection items and has a sound interface that co-operates with the operation interfaces of the control unit to simultaneously sound the selected operational selection items and the operational function; and
the therapeutic apparatus further comprises a plurality of filtering layers mounted in the air inlets of the housing.

3. The therapeutic apparatus of claim 1, wherein the ceramic portion of the electric heating element is heated and rectified by the circuit module to have a predetermined temperature ranged between 25 and 150° C., and the air delivery and regulating unit regulates an ambient cold air at a predetermined flow rate ranged between 0.1 and 5 L/min.

4. The therapeutic apparatus of claim 1, wherein the fixture is mounted on the air outlet of the housing and is provided with a contact head that is available for the nose, the mouth or the toes of the human body.

5. The therapeutic apparatus of claim 1, wherein:
the therapeutic apparatus further comprises an atomized vapor humidification unit electrically connected with the circuit module and the control unit;
the atomized vapor humidification unit co-operates with the air delivery and regulating unit so that an atomized vapor from the atomized vapor humidification unit is mixed with the hot air flow from the electric heating element to flow through the air channel and the air outlet of the housing into the fixture.
